(19) United States
(12) Patent Application Publication
(10) Pub. No.: US 2023/0141834 A1
(43) Pub. Date: May 11, 2023

(54) DRYER

(71) Applicant: Kohler Co., Kohler, WI (US)

(72) Inventors: Peter Kajuch, Brookfield, WI (US); William Kuru, Plymouth, WI (US); Peter Denzin, Glenbeulah, WI (US); Jason Kwacz, Kohler, WI (US)

(21) Appl. No.: 17/975,077

(22) Filed: Oct. 27, 2022

Related U.S. Application Data
(60) Provisional application No. 63/278,372, filed on Nov. 11, 2021.

(51) Int. Cl. A47K 10/00; F26B 21/00; A61L 2/00

(52) U.S. Cl. CPC A47K 10/00; F26B 21/004; A61L 2/0047; A61L 2/0088; A47K 2210/00

(57) ABSTRACT

A hand dryer apparatus includes a fan, a duct, at least one air knife driven by the fan and configured to direct air to dry one or more hands and push water from the one or more hands into the duct, and at least one cyclone device to provide at least a partially circular path for the air and water from the hand.

This application claims priority benefit of Provisional Application No. 63/278,372 filed Nov. 11, 2021, which is hereby incorporated by reference in its entirety.

### FIELD

The present application relates generally to hand dryers.

### BACKGROUND

Hot air operated hand dryers have been available for over half a century. In recent decades, the major advancement has been high velocity air jets which can substantially dry hands in 10-15 seconds even without adding heat. This is accomplished by the force of air stripping water from the skin, mostly mechanically rather than by evaporation. The energy, cost, and cleanliness compared to paper towels has been researched, debated and published in a variety of articles. Both have unique advantages, hence both jet dryers and paper towels exist based on preferences or biases. For example, in some studies, the energy and cost of using jet dryers was considerably lower than paper towels. In other cases, the initial cost of jet dryers may be a hurdle. Specific hygienic concerns of a hospital or waste management concerns of an arena or small establishment may influence the decision to use a jet dryer or paper towels.

In very recent times, the concern about airborne microbes has become heightened. The hygiene of jet dryers is being debated based on the perception that high velocity air jets can detach microbes from surfaces and significantly mobilize germs in a room. The research and publications are divided on proving this concern, but the possibility is real.

Most of the conventional devices deliver air jets onto the hands from a perpendicular direction causing water and air to splash in every direction including onto a wall and onto the user. Conceivably, splashing can initiate biofilm formation and promote growth on surfaces, and air jets deflected by hands could dislodge biofilms. Furthermore, most of the air jets are delivered into the washroom space with no substantial containment, thus increasing the fear about spread of germs.

### BRIEF DESCRIPTION OF THE DRAWINGS

Exemplary embodiments are described herein with reference to the following drawings, according to an exemplary embodiment.

FIG. 1 illustrates an example hand dryer.

FIG. 2A illustrates example cyclones for the hand dryer of FIG. 1.

FIG. 2B illustrates a top view of the hand dryer of FIG. 1.

FIG. 3A illustrates an example air knife for the hand dryer of FIG. 1.

FIG. 3B illustrates an example air knife for the hand dryer of FIG. 1.

FIG. 4A illustrates a position of the air knife with respect to a duct of the hand dryer.

FIGS. 4B and 4C illustrate a position of the air knife with respect to a horizonal.

FIGS. 5A and 5B illustrate another embodiment of a hand dryer.

FIGS. 6A and 6B illustrate another embodiment of a hand dryer.

FIG. 7 illustrate a cross section of the hand dryer of FIGS. 6A and 6B.

FIGS. 8A and 8B illustrate another embodiment of a hand dryer including an expansion chamber.

FIG. 9 illustrates another arrangement of air knives for a hand dryer.

FIG. 10 illustrates another arrangement of air knives for a hand dryer.

FIGS. 11A and 11B illustrate another arrangement of air knives for a hand dryer.

FIGS. 12A and 12B illustrate a circular air duct with a vacuum source for a hand dryer.

FIGS. 13A and 13B illustrate another embodiment of a hand dryer.

FIGS. 14A and 14B illustrate another embodiment of a hand dryer.

FIGS. 15A and 15B illustrate another embodiment of a hand dryer.

FIG. 16 illustrates an example hand dryer according to any of the embodiments herein mounted in association with a sink.

FIG. 17 illustrates a cutaway view of the example of FIG. 16.

FIG. 18 illustrates multiple hand dryers according to any of the embodiments herein integrated with a sink.

FIG. 19 illustrates multiple hand dryers according to any of the embodiments herein integrated with a sink and cabinet.

FIG. 20 illustrates an example controller for any of the dryer systems of FIGS. 1-19.

FIG. 21 illustrates an example flow chart for the controller of FIG. 20.

### DETAILED DESCRIPTION

The following embodiments include air dryers (e.g., hand dryers) that include a ducted cavity and at least one air knife angled into the ducted cavity. The ducted cavity, as well as the angle and orientation of the at least one air knife, is arranged to apply the jet or jets of air to a single hand of the user. The at least one air knife may be provided in a vertical direction to apply to the single hand of the user in a “handshake” position. In this way, the air is provided to both sides of the hand simultaneously. Some examples include a cyclone device in which the air in the ducted cavity rotates for the removal of water droplets, aerosols, or other particles. The particles may be propelled against the walls of the cyclone device, where they can be easily removed, sanitized, disinfected, or otherwise cleaned. The cyclone may connect to an exhaust path so that the air, water, aerosols, and other particles are provided to another space (i.e., away from users in the bathroom). Additional examples include an expansion chamber to slow the flow of air in the ducted cavity. These components, arranged in this manner to dry a single hand of the user, operate with low power requirements. The air dryer may user smaller motors and fans than similar two-hand dryers. In addition, the vertical arrangement (“handshake” position of the hand) allows for versatility in both the height of the air dryer (mounting position) and the height of the user.

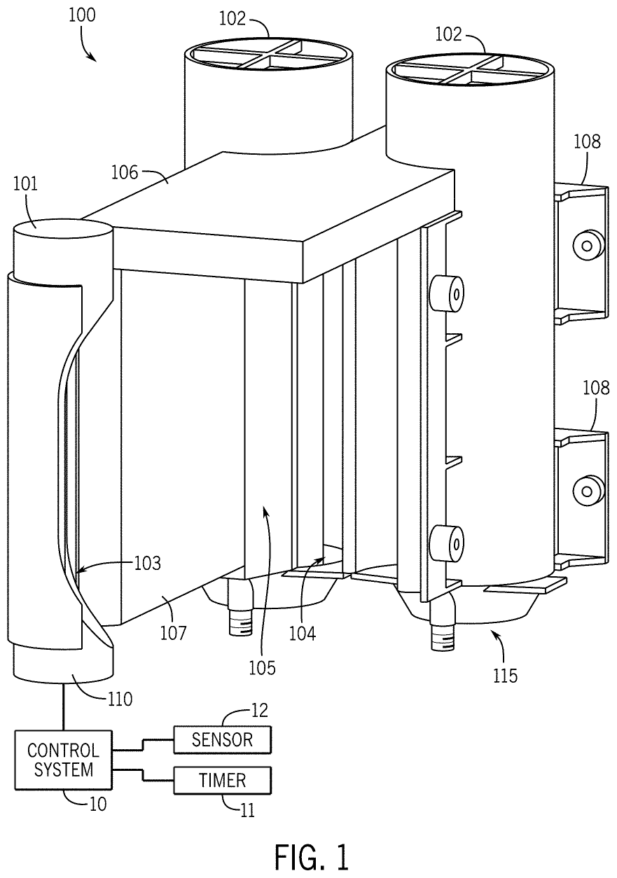

FIG. 1 illustrates an example hand dryer or air dryer system 100 including at least one air knife 101 and a ducted cavity. The air knife 101 may include an aperture 103 and a fan 110. The fan 110 generates an airflow through the hollow portion of the air knife 101, which tapers to the thin aperture 103, causing an increase in the velocity of the air flow. The fan 110 may generate a predetermined flow rate such as 25 cubic feet per minute or greater. As an alternative to the aperture 103, a series of holes may be used. The fan 110 may be separated from the air knife 101 through a hose or sealed passage.

As an example of the operation principle of an air knife, when used in manufacturing settings, an air knife may be mounted along a conveyer belt on which a product or other object travels. The air knife emits a high-intensity, uniform sheet of laminar airflow to dry the objects (i.e., mechanically strip or remove water from the objects). In the enclosed embodiments, the air knives direct such a uniform sheet if laminar air flow onto an object such as the user's hands that are held in the air flow. The hands may be moved in a particular pattern or direction (e.g., into the dryer, in a vertical plane, in a vertical plane and away from the user, in a vertical plane and into the dryer, in a direction perpendicular to the air flow, or another direction). An indicia on the outside of the hand dryer may instruct the user in the particular pattern or direction.

The ducted cavity may include at least one cyclone device 102 or cyclonic device and an inlet air path between the air knives 101. The air inlet path may be defined on each side by a side wall 107 and on the top wall 106. Thus, the inlet air path may be defined on three sides, including two side walls 107 and the top wall 106. An open space or gap may be present between lower portions of the air knives 101. It should be noted that no bottom wall is included in certain embodiments. As illustrated in FIG. 1, only one side wall 107 on the left side is illustrated, but a symmetrical side wall 107 may also be present on the right side as well. The side walls may be different shapes and inclined in any direction. The cyclone devices 102, or an associated housing, may be secured to a wall or other structure by brackets 108 via screws, bolts, or other fasteners. The space between the air knives 101 may be open on the bottom (that is not bottom wall). This is because water is substantially prevented from dripping due to the orientation of the air knives. Any water that does drip is allowed to fall freely, thus avoiding any pooling water or accumulated moisture as much as possible. The cyclone devices 102 may include a drain 115 (e.g., a nozzle and/or pan) for accumulation of water and/or expulsion of water from the cyclonic devices 102. Additional, different, or fewer components may be included.

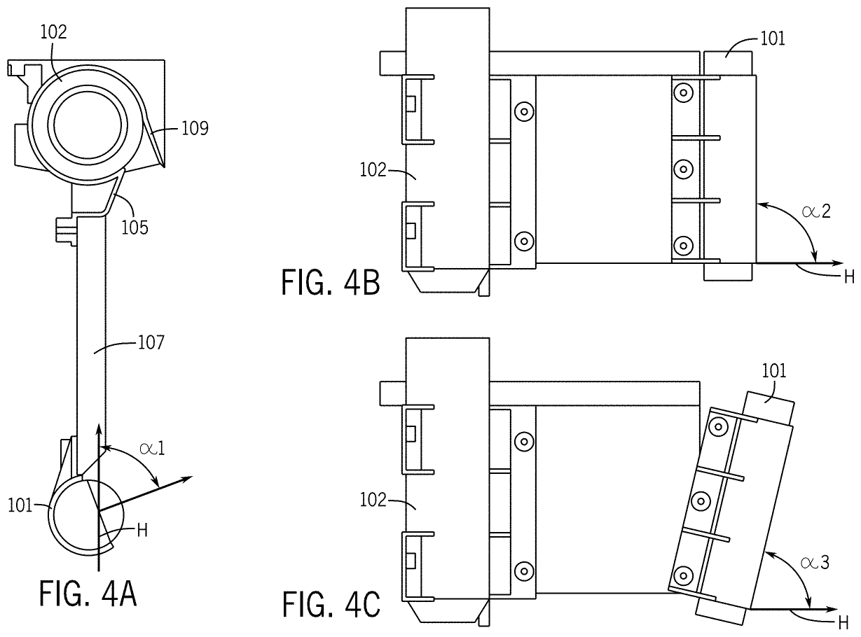

The ducted cavity may also be defined by an entry plate 105 that connects the cyclone device 102 to the side wall 107. The entry plate 105 may be coupled to the cyclone device 102 via a fastener or adhesive. A divider 109 (shown in FIG. 4A) in one or more of the cyclone devices may form cyclone inlet 104. Air from the air knives 101 flows into the cyclone device 102 through the cyclone inlet 104.

Between the side walls 107 is a drying space where an object is placed between the air knives 101 for drying. The object may be one or more of a user's hands. The hands may be placed at a predetermined angle, which is guided by the shape and orientation of the drying space. The air knives 101 may be mounted at a predetermined angle (e.g., a predetermined angle in up to three directions or measured from any combination of three axes) that optimizes or maximizes the drying of the object. The air knife 101 driven by the fan 110 directs air to dry one or more hands and push water from the one or more hands into the ducted cavity.

The hand dryer 100 may be configured to dry a single hand at a time. The space between the air knives 101 may be narrow and sized for a single hand. The air knives 101 provide the jets of air to the two sides of the single hand simultaneously. In some examples, one hand is placed in the hand dryer 100 for a time period and then the other hand is placed in the hand dryer 100 for a time period. In some embodiments, the system includes two hand dryers 100 placed at a comfortable distance a part so that a left hand is placed in the left hand dryer at the same time that a right hand is placed in a right hand dryer.

The hand dryer 100 is shaped and orientated so that the exhaust air that is expelled by the hand dryer 100 is captured by the cyclones 102 to separate water and slow the air down to be exhausted away from the user. The orientation of the air knives 101 and drying cavity allows the user to place the user's hands substantially straight out using ergonomics similar to that of a handshake. The air knives 101 may be oriented so that no water splashes outside of the hand dryer 100, as described through the disclosed embodiments.

A controller 10 may send commands, provide power to, or otherwise operate the fan 110 for driving the air knife 101. The controller 10 may be couped to a sensor 12. The sensor 12 is configured to generate sensor data for an object in vicinity to the hand dryer. The sensor 12 may be a proximity sensor that detects an object, such as the user's hands in proximity to the hand dryer. For example, the sensor 12 may detect the user's hands within a predetermined distance to the air knife 101 or within the drying space. The sensor 12 may detect another object or a gesture made by the user. In some examples, the sensor may include any type of sensor configured to detect certain actions. A proximity sensor may be employed to detect the presence of an object within a zone of detection without physical contact between the object and the sensor. Electric potential sensors, capacitance sensors, projected capacitance sensors, light detection and ranging (LiDAR), and infrared sensors (e.g., projected infrared sensors, passive infrared sensors) are non-limiting examples of proximity sensors that may be employed with the systems of this application. Motion sensors may be employed to detect motion (e.g., a change in position of an object relative to the objects surroundings). Electric potential sensors, optic sensors, radio-frequency (RF) sensors, sound sensors, magnetic sensors (e.g., magnetometers), vibration sensors, and infrared sensors (e.g., projected infrared sensors, passive infrared sensors) are non-limiting examples of motion sensors that may be employed with the systems of this application. In another example, the sensor may include a time of flight (ToF) or a LiDAR that servers as a proximity sensor. The controller 10 receives sensor data and analyzes the sensor data to determine when a user is approaching or has approached the hand dryer. The controller 10 turns on the air knife 101 and/or fan 110 in response to the analysis of the sensor data. A mechanical button, switch, or sensor may be used rather than a touchless sensor.

The controller 10 may implement a timer or be coupled to a timer 11. The timer may count to an elapsed time period. The time period may be an amount of time after the user's hand, or another object are no longer detected by the sensor 12 before the controller 10 instructs the fan to turn off. In one example, the controller 10 may also turn off if a maximum time limit is reached by the timer 11 since the fan 110 was turned on.

In some examples, the controller 10 turns on the air knife 101 and/or fan 110 turns on in response to the detection of the user's hand(s) by the sensor 12 and turns off the air knife and/or the fan 110 in response to the elapsed time passing after the user's hand(s) are no longer detected. Thus, the controller 10 is configured to operate the fan 110 to move air through the hand dryer in response to the sensor data or the elapsed time period. The controller 10 may start the fan to in response to the sensor data and stops the fan in response to the elapsed time period.

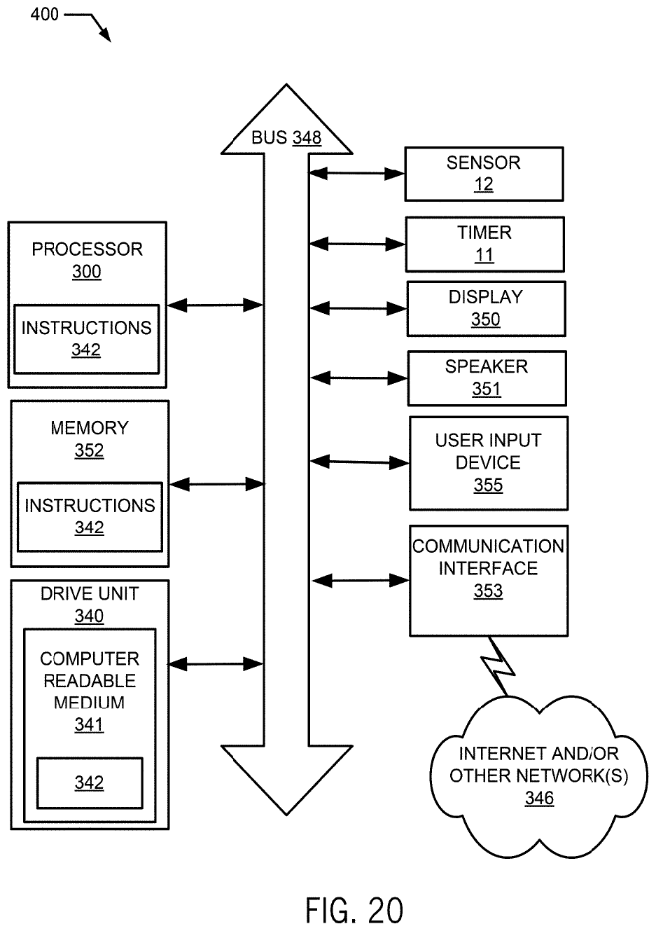

The controller 10 may operate in a low flow rate mode to clean the room air. For example, even when no user's hands or objects are present in the dryer space, the controller 10 may operate the fan 110 to circulate air from the room into the hand dryer for any of the disinfecting, sanitization, or cleaning techniques described herein. The controller 10 may start the low flow rate mode at a predetermined time (e.g., at 2 AM or other overnight time period, or during a weekend) as determined by the timer 11. The controller 10 may be loaded with a schedule or calendar for the low flow rate mode. An external button (e.g., user input device 355, FIG. 20) may trigger the low flow rate mode. In some examples, the sensor 12 includes an air quality sensor and the controller 10 triggers the low flow rate mode in response to data from the air quality sensor.

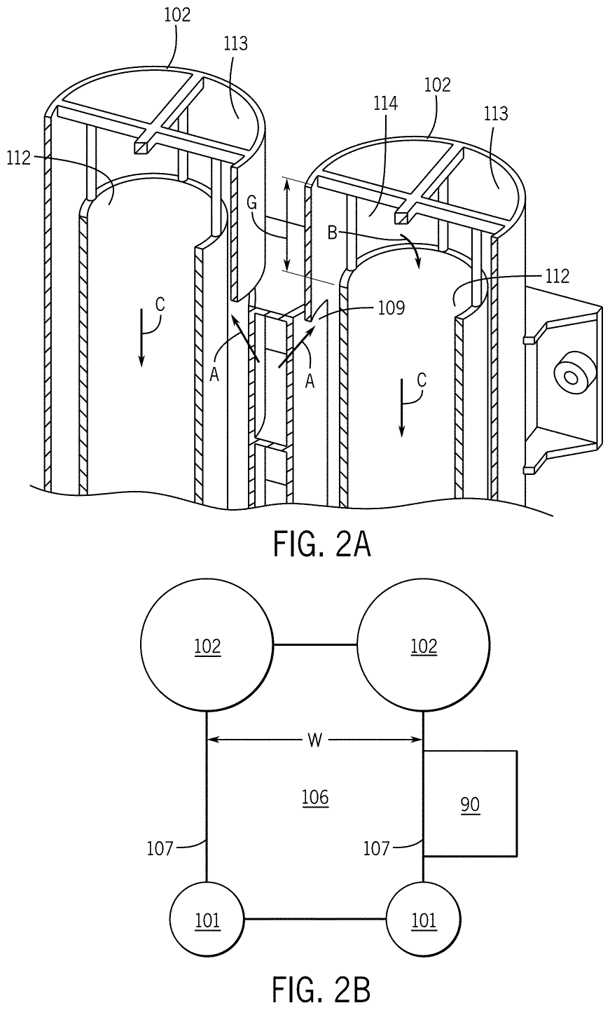

FIG. 2A illustrates example cyclones 102 for the hand dryer of FIG. 1. The air and water is driven through the ducted cavity and around the cyclones 102, which applies a force to the water including aerosols or other particles to the outside of the cyclones 102. As discussed in more detail below, the particles adhere to the radial surfaces of the cyclones 102.

In some examples, the cyclones 102 are covered or otherwise enclosed on the top and air is vented to escape through the bottom of the cyclones 102 (as shown in FIG. 2A). In other examples, the cyclones 102 are covered or otherwise enclosed on the bottom and air is vented to escape through the top of the cyclones 102. In other examples, the cyclones 102 may be vented on both the top and the bottom so that air can escape on both the bottom. In addition or in the alternative, vents may be included on the sides of the cyclones. The vents may direct the air to another room, inside the wall, or a ventilation system.

The cyclones 102 may include two concentric channels including an inner channel 112 and an outer channel 113. The cyclones 102 may be formed of two cylinders such that the inner channel 112 passes through the inside of an inner cylinder and the outer channel 113 passes between the inner cylinder and the outer cylinder. Air passes from the air knife 101 into the ducted cavity and past the divider 109 as shown by arrow A into the outer channel 113. One or more holes or windows 114 connect the outer channel 113 to the inner channel 112. As shown by arrow B, the air flows through the windows 114 from the outer channel 113 into the inner channel 112. A gap G defines the height of the windows 114 or a distance between the edge of the inner channel 112 to the end plate of the cyclone 102. The gap G may be varied to regulate the volume of air (e.g., flow rate or speed) flowing from the outer channel 113 to the inner channel 112. As described below, the gap G and associated flow rate may be selected according to disinfection technique, or another treatment applied to the air in the inner channel 112. As shown by arrow C, the air the flows through the inner channel 112 to the vent.

The inner cylinder forms a baffle that forces the air flow to at least partially flow around the inner cylinder in at least a partially circular path for the air and water from the user's hand. The term circular may describe the cross-section of the inner cylinder and/or the outer cylinder. The term circular may describe the up and down or serpentine path through the inner and outer channels.

The inner cylinder forms a baffle that forces the air flow to at least partially flow around the inner cylinder in at least a partially circuitous path for the air and water from the user's hand. The term circuitous may describe the change in direction from the inner cylinder to the outer cylinder. Other shapes besides cylinders may be used. That is, the inner cylinder and the outer cylinder may be rectangular, square, oval, or another shape in cross section.

In addition, or in the alternative, other baffles such as in the radial or longitudinal direction with respect to the inner cylinder. Other flaps, channels, labyrinths or passages may be included to ensure that the path of the air is long enough for the water droplets and aerosols to be removed by centrifugal force. The particles expelled from the air and water adhere to cyclone device 102. In some examples, the inside surface of the cyclone device 102 may be textured to facilitate the adherence. In some examples, the moisture that accumulated on the inside surface of the cyclone device 102 facilitates the adherence.

Other examples are possible for the construction for the cyclones 102 may include another number of concentric channels. Three channels, four channels, or more may be utilized. In some example, the channels have different heights. That is, one of the channels may be a proportion (e.g., half) of the height of one or more other channels.

In one example, the air flows from the duct to a first outer channel. Air passes from the air knife 101 into the ducted cavity and past the divider 109 as shown by arrow A into the outer channel. From the outer channel, air passes to a first inner channel through one or more windows or apertures. From the first inner channel, air passes to a second inner channel through one or more windows or apertures. Any number of channels may be used. The channels may have a variety of heights. The channels may have a variety of relative diameters or widths. For example, the first inner channel may have a diameter that is a predetermined proportion or percentage of the outer channel (e.g., 80%) and the second inner channel may have a diameter that is a predetermined proportion or percentage of the first inner channel (e.g., 80%).

In some examples, the air flow from the duct first flows upward through the outer channel 113 into the inner channel 112 and downward through the inner channel 112. In other examples, the air flow from the duct flows downward through the outer channel 113 into the inner channel 112 and upward through the inner channel 112. In the case of three channels, the air flow may substantially travel upward through the outer channel, downward through the first inner channel, and upward through the second inner channel. Alternatively, the air flow the air flow may go downward through the outer channel, upward through the first inner channel, and downward through the second inner channel.

Once the aerosols or other particles are adhered to the inside surface of the cyclone device 102, one or more disinfectants or disinfecting techniques are applied to the particles within the cyclone device 102.

In one example, a light such as ultraviolet light is mounted in the cyclone device 102, or adjacent to the cyclone device 102 through a window. The ultraviolet light irradiates the internal walls. The ultraviolet light may have a predetermined frequency or wavelength, which may be a range of wavelengths or frequencies for the light emitted from the light. The germicidal irradiation may be optimized by a wavelength band of 200 to 280 nanometers (nm) other examples may include 200 to 222 nm, 230 to 250 nm, 240 to 315 nm or other ranges. An example wavelength may be 254 nm. The controller 10 may send commands to the light to turn on the light or stop the light. The controller 10 may send commands to the light to set the wavelength of the light. The ultraviolet light disinfects the particles. The ultraviolet light may kill or eliminate living organisms (e.g., bacteria) and/or viruses that are adhered to the inside surface of the cyclone device 102 or otherwise contained in the cyclone device 102 (e.g., in a mist). Ultraviolet light may be run for at least 30 seconds after a user has finished using the hand dryer. In high use cases ultraviolet light may be run continuously. This option may be set up by the building operator, or it may be done by machine learning or other artificial intelligence (AI).

In one example, a liquid or suspended disinfectant may be sprayed or dispersed into the cyclone device 102. The disinfectant may be hydrogen peroxide (H₂O₂), chlorine, citric acid, electrolyzed water, or ozone (O₃). The hydrogen peroxide may be stored in a tank that is refilled by the user or a service technician. The ozone may be generated by a corona charger that ionizes the air in or around the cyclone device 102 using a high voltage that causes the air to breakdown and become conductive. The corona occurs when the potential gradient of the electric field around the charger is greater than the dielectric strength of the air. When ozone is used there is an option of adding ultraviolet (UV) decomposition phase after ozonation. A short UV irradiation phase will decompose the ozone and reduce the amount of ozone that escapes the hand dryer.

The gap G between the outer channel 113 and the inner channel 112 may be set according to the type of treatment. In one example, treatment from UV light may be associated with a lower flow rate (larger gap G) and treatment from a sprayed or atomized mist may be associated with a higher flow rate (smaller gap G).

The controller 10 may operate a disinfectant dispenser configured to provide the disinfectant to the cyclone device 102. The dispenser may include a nozzle or sprayer that may be electronically actuated by the controller 10. The controller 10 may operate the charge to generate the ozone within or adjacent to the cyclone device 102.

The controller 10 may operate an ultrasonic emitter to provide ultrasonic waves to the cyclone device 102. The ultrasonic emitter may include an ultrasonic atomizer or transducer that converts high frequency sound waves into mechanical energy that is transferred into standing waves of the sanitizing liquid, causing a mist or fog to be emitted.

The controller 10 may operate in a sanitation mode to release a sanitizer into the hand dryer. The sanitation mode may occur after the drying mode. For example, after a predetermined has elapsed from drying, the sanitation mode is started by the controller 10. During the sanitation mode, any of these techniques (e.g., ultraviolet light, ozone generation, disinfectant dispensing, ultrasonic wave generation) may be performed under commands from the controller 10. The sanitation mode may be performed at periodic intervals or at predetermined times of day or days of the week. The sanitation mode may be performed in response to the sensor data (i.e., after the drying mode) and/or in response to an elapsed time period (i.e., a certain amount of time after the drying mode has started or ended).

FIG. 2B illustrates a top view of the hand dryer of FIG. 1. FIG. 2B illustrates that the cyclone devices 102 are to the rear of the air knives 101 and the top plate 106. The cyclone devices 102 and the air knives 101 may be fastened or adhered (e.g., glued) to the top plate 106.

A predetermined distance, or dryer width W, defines a distance between the side walls 107 or between the centers of the air knives 101. The width W may be width of the ducted cavity. The width may define the proximity of the air knives 101, and corresponding air jets, to the one or more hands. The distance between the air knives 101 and the one or more hands impacts the speed and effectiveness of the air to remove water from the one or more hands. It is beneficial to cause the user to bring the one or more hands as close as possible to the air knives 101 while also providing sufficient space for relatively large hands and at the same time providing enough space for the user to easily avoid touching the sides of the ducted cavity. In several embodiments, the width is selected for a single hand so that the single hand is in close proximity to both air knives 101 but far enough apart for the user to maintain a comfortable distance between the air knives 101. A range for the width W is 2 to 4 inches or 2.750 to 3.125 inches. An example selected width W may be 3 inches.

In any of the examples described herein, one or more filters may be included upstream of the hand dryer, within the hand dryer, and/or downstream of the hand dryer. The filtering may be provided in addition to or in the alternative of the sanitization and disinfection techniques described herein. The upstream air filter may be coupled to the fan so that all air traveling through the fan has been filtered. The filter within the hand dryer may be upstream of the air knife 101, in the ducted cavity, or in the cyclone device 102. The filter downstream of the hand dryer may be at the air exhaust of the hand dryer. Any of these filters, to the extent room air is circulated, may be a room filter configured to filter air in the vicinity of the apparatus.

Any of these filters are configured to remove particles from the air. The filter may be a pleated mechanical air filter such as HEPA (high efficiency particulate air filter). The filter may be a separation filter based on particle size. The filter may include activated carbon.

The filter may be an electrostatic separator. For example, an electrostatic aerosol collector is biased with a voltage to provide the electrostatic charge. The voltage may be a low voltage that avoids the risk of shock. In some embodiments, the electrostatic aerosol collector is charged through the physical properties of the material. In some embodiments, electrostatic aerosol collector is charged through frictionally moving two components together. In order to maintain the electrostatic charge on the plastic sheet, the sides, edges, or corners may be insulated. The insulation may include non-conductive materials between the plastic sheet and the wall or other devices.

In any of these examples a hand disinfectant dispenser 90 may be included adjacent to or coupled with the hand dryer. The hand disinfectant dispenser 90 may be automatically (e.g., by electronic control from the hand dryer controller or a proximity sensor) or manually (e.g., by push button or gesture) actuated to dispense disinfectant on the hands of the user. When automatically controlled, the hand disinfectant dispenser 90 may be actuated before, during, or after the fan 110 is actuated.

In a first example, the controller 10 may receive sensor data indicative of a user is approaching or has approached the hand dryer, and the controller 10 turns on the hand disinfectant dispenser 90 before turning on the air knife 101 and/or fan 110. In a second example, the controller 10 may receive sensor data indicative of a user is approaching or has approached the hand dryer, and the controller 10 turns on the hand disinfectant dispenser 90 simultaneous (or near simultaneous within a predetermined time period) to turning on the air knife 101 and/or fan 110. In a third example, the controller 10 may receive sensor data indicative of a user is approaching or has approached the hand dryer, and the controller 10 turns on the hand disinfectant dispenser 90 after turning on the air knife 101 and/or fan 110, after turning off the air knife 101 and/or the fan 110, or after a predetermined time delay.

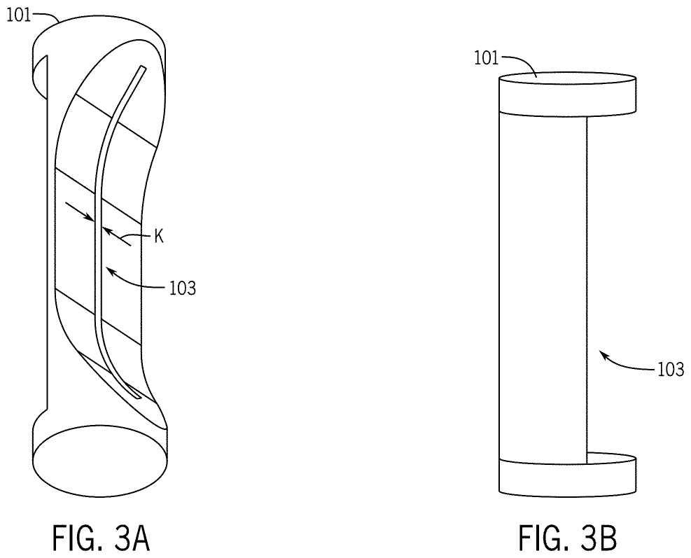

FIG. 3A illustrates an example air knife 101 for the hand dryer of FIG. 1 having a curved or angled aperture 103. The aperture 103 may have a predetermined width or air knife gap K. Examples for the gap K may be in the range of 0.01 to 0.05 inches. One example gap K is 0.03 inches. The size of the gap K affects the speed and force of the air. Smaller gaps greater an air knife with a higher force that quickly strips water from a hand. However, if the gap K is too small, and the corresponding force is too high, the air knife may feel too strong to the user.

FIG. 3B illustrates an example air knife 101 for the hand dryer of FIG. 1 having a straight or linear aperture 103. The linear aperture 103 may also have a selectable or variable aperture 103 having gap K. The gap K may be varied by an adjustment screw that brings one plate of the air knife 101 closer together or father apart from a second plate of the air knife 101.

FIG. 4A illustrates a top down view of the hand dryer including a position of the air knife 101 with respect to a duct of the hand dryer. For example, FIG. 4A illustrates that an angle α1 of the air knife aperture 103 may be angled at an acute angle with respect to the side wall 107, which may be aligned with a horizontal plane H. In one example, the angle α1 between the knife aperture 103 and the horizontal plane may be in a range of 45 to 60 degrees. One specific example angle α1 may be 55 degrees. The angle α1 may be selected to maximize the amount of air that is directed into the duct of the hand dryer. When the angle α1 is too low the intersection point of the air flows from the knife apertures 103 is too far inside the duct to effectively strip water from the same object (user's hand). When the angle α1 is too high, air may be deflected from the wrist of the hand. Higher angles may also result in resonant tones and vibrations.

As the predetermined angle is increased, the air knife 101 is pointed more into the ducted cavity to push the air and water into the dryer but less direct drying force is applied to the user's hands. The angle may be selected to maximize the speed and effectiveness of drying as well as forcing the air and water into the dryer.

The predetermined angle (e.g., 55 degrees) may act to self-center the user's hand(s) in the hand dryer. The flow of air from the air knives 101 may apply forces to the user's hand(s) that are substantially balanced. Shorter angles may cause forces having a larger perpendicular force against the user's hand(s) that tends to push the user's hand(s) against the side wall 107.

FIGS. 4B and 4C illustrate a position of the air knife 101 with respect to a horizonal. The incline, or angle with the horizontal plane, of the air knife 101 may affect the angle that water is pushed off the user's hands. When the angle with the horizontal plane is at a first angle α2 which may be substantially perpendicular (e.g., 90 degrees), drying may be maximized when the user moves hands at an angle. When the angle with the horizontal plane is a second angle α3, which may be an acute angle (e.g., 70 degrees), drying may be maximized when the user moves hands up and down.

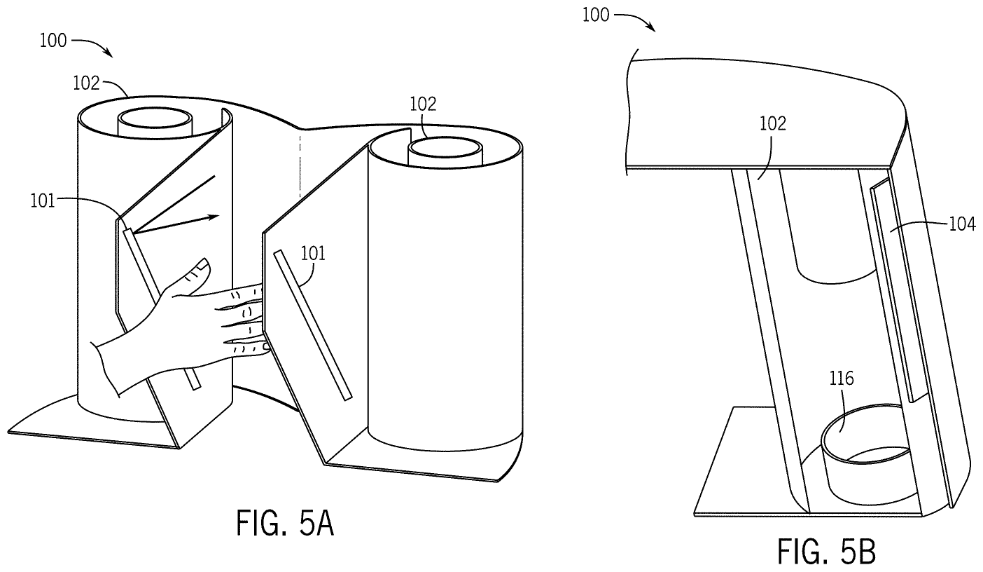

FIGS. 5A and 5B illustrate another embodiment of a hand dryer. In this example, the air knives 101 are inclined toward the front of the hand dryer 100 and the apertures 103 are pointed inward to push air and water into the cyclones 102. In this example, a single construction (e.g., molded material or deformable material) is shaped to form integrated cyclones 102 and air ducts, including at least one cyclone input 104 and at least one cyclone output 116. The cyclone output 116 may open into a space below the hand dryer 100. The cyclone output 116 may be connected to a tube or duct that directs the exhaust air to a predetermined location. The cyclone output 116 may be exhausted to another room or into a heating or ventilation system.

FIGS. 5A and 5B further illustrate the vertical arrangement of the air knives 101 allowing the “handshake” orientation of the single hand in the vertical plane so that both air knives 101 provide air jets to the single hand. The air knives 101 are inclined down and away from the user as well as pointed in so that the water stripped from the single hand is pushed immediately toward the cyclones 202. Because the air knives 101 are on opposite sides of the hand, water does not “roll” from one side of the hand to the other. Instead, the water is pushed forward into the cyclones 202. Because of the vertical space for the single hand to be inserted into the hand dryer 100, users of varying heights, even users that may need to reach up or above their heads to reach the hand dryer 100, can comfortably place hands in the vertical plane between the air knives 101. For similar reasons, the hand dryer 100 may be mounted at a variety of heights while accommodating all users.

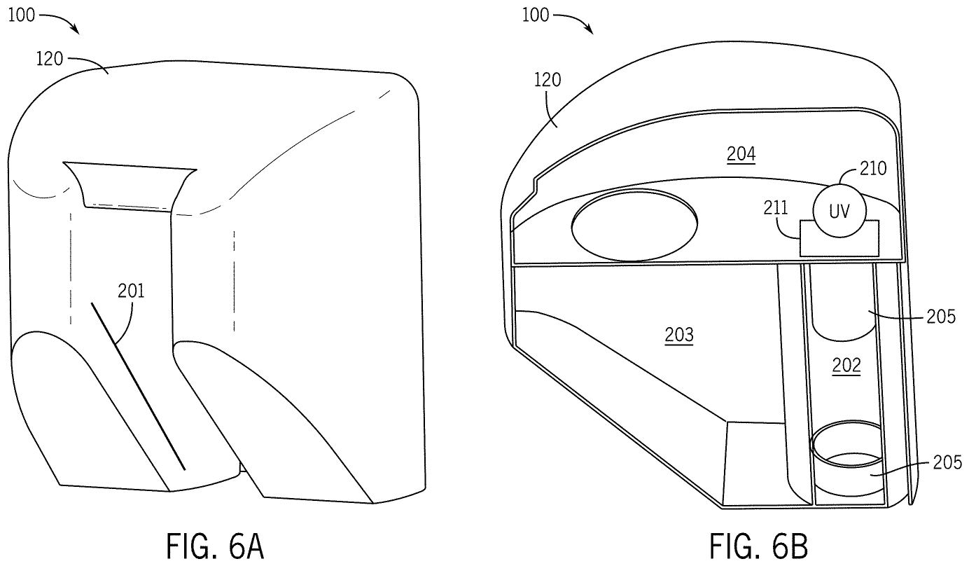

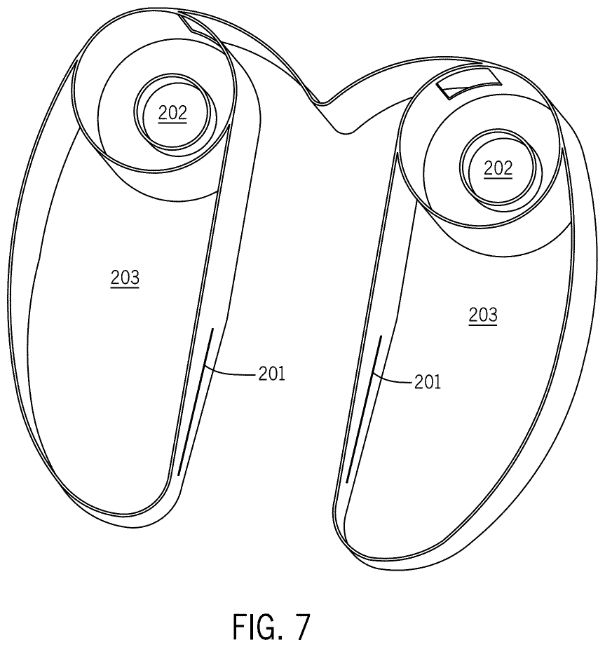

FIGS. 6A, 6B, and 7 illustrate another embodiment of a hand dryer 100. FIG. 7 illustrates a cross section of the hand dryer of FIGS. 6A and 6B. The hand dryer 100 of this embodiment may include any of the components of other embodiments described herein. This hand dryer may include vertical space so as to be operable to dry one hand or two hands simultaneously. The hand dryer includes a housing 120 including one or more air jets 201 with corresponding pressure chambers 203 connected to a fan chamber 204. Air from the air jets 201 enters a drying chamber for drying the user's hands and then enters a cyclonic separator 202. Additional, different, or fewer components may be used.

Referring to FIG. 6A, the air jets 201 are angled to sweep over hands as they enter and exit the slot of the drying space horizontally or vertically. Further the air jets 201 are directed toward the interior of the duct away from the user. Referring to FIG. 6B, the air jets 201 are supplied by a common fan supplying the pressure chamber 203. As the air jet is deflected from the hand, it is directed toward the interior wall which divides the flow into two passages. Each of the passages are the inlet to the cyclonic separator 202.

A window 211 may provide an optical path between the fan chamber 204 and the cyclonic separator 202. An ultraviolet light 210 may be mounted in proximity to the window 211. The ultraviolet light 210 may transmit UV light into the cyclonic separator 202 to disinfect the air and water traveling through the cyclonic separator and received from the drying duct.

Referring to FIG. 7, the air flow enters through a slot in the cyclonic section 202, and after several turns exits through an opening directed toward the floor. The cyclonic section 202 separates air and water and slows the velocity of exhaust. The cyclonic section 202 has an upturned air exit to trap water which can be routed to a drain or collected in a container.

For the example of a single-handed device, the hand dryer may dry objects rapidly by using high velocity air jet, but the motor may be smaller, thus produce less noise. Further, requiring less volume flow can reduce the mobilization of microbes. In addition, the complete device can be smaller and lower in cost.

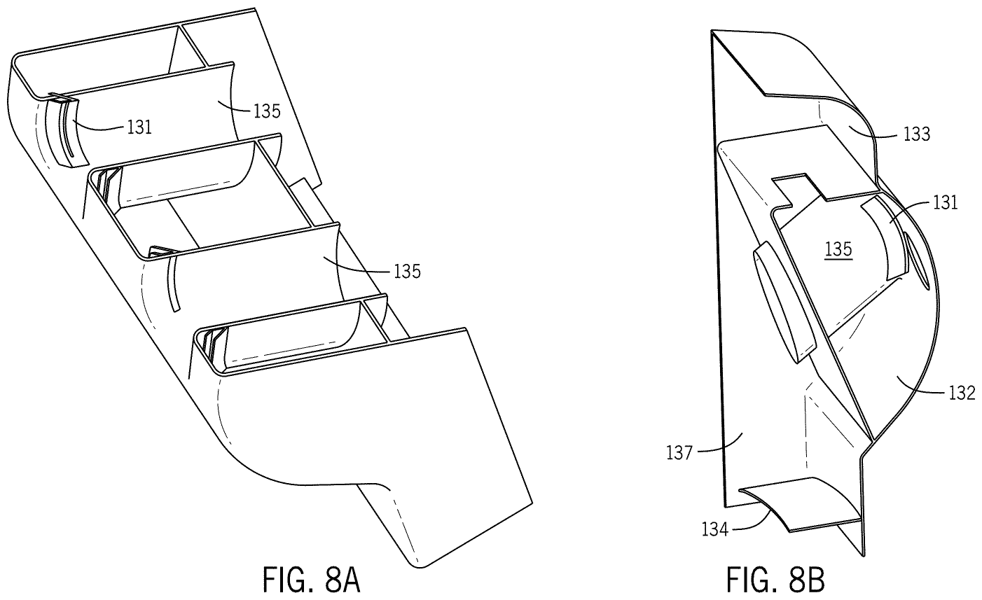

FIGS. 8A and 8B illustrate another embodiment of a hand dryer with a ducted dryer for drying two hands simultaneously. The hand dryer includes a plurality of air knives 131 arranges on multiple drying spaces 135 or ducts (e.g., a first space 135 for a left hand and a second space 135 for a right hand). The air knives 131 may be angled inward (e.g., 55 degrees) to the spaces 135. The hand dryer includes a fan section 133 including a fan and a pressure chamber 132 that is pressurized by the fan to provide the air flow to the air knives 131. Out of the drying spaces 135, the air and any suspended water, aerosols, or other particles are provided to an expansion chamber 137, with an increased volume that slows down the flow.

For the hand dryer of FIGS. 8A and 8B, the hand orientation is vertical and arm orientation is extended with slight bend at the elbow. These ergonomics provides for a range of user heights. The ducts are sized to accept hands larger than 99th percentile male. Each duct delivers high velocity air jets to both sides of the hands. The air jets are angled into the duct such that the jet will impinge on the hands with sufficient pressure to remove water while also deflecting further into the duct. This action eliminates splash back onto the user. The direction of the air jet or air knife 131 is controlled by nozzles in a pressure chamber 132 supplied by a common fan. After the air passes through the duct, it enters an enclosed chamber (e.g., expansion chamber 137) of significantly larger volume to slow the flow velocity. A diffuser 134 may be configured diffuse the flow to separate air and water and further slow the velocity. The final exit is directed downward to the floor away from the user. The separated water can be collected and routed to a drain or container.

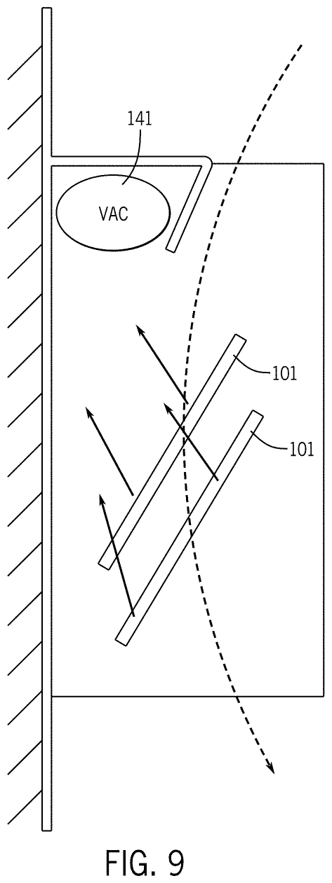

FIG. 9 illustrates another arrangement of air knives 101 for a hand dryer. In this example, two air knives 101 may be arranged in parallel one behind the other. A vacuum 141 may draw the air from the air knives 101 across the drying space into the ducted cavity. A hand dryer with two or more air knives 101 may be faster, dryer, utilize a lower air velocity, create less noise, and produce more air entrainment to protect a user. With individually controllable air knives, different velocities may be selected (e.g., low air, high air). Higher velocities for the outer knives help to remove water from the skin, and higher flow rates on the inner knives carry droplets to the collection system.

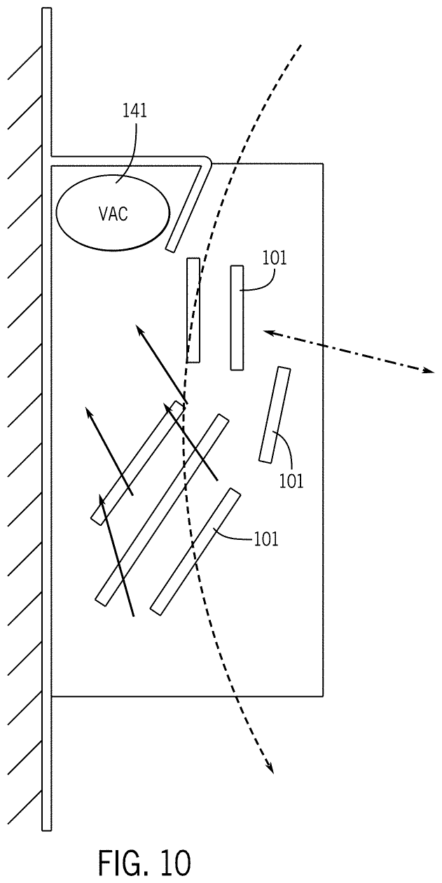

FIG. 10 illustrates another arrangement of air knives 101 for a hand dryer. In this example, multiple (e.g., five) air knives 101 may be arranged in varying angles and generally directed to an internal point toward vacuum 141.

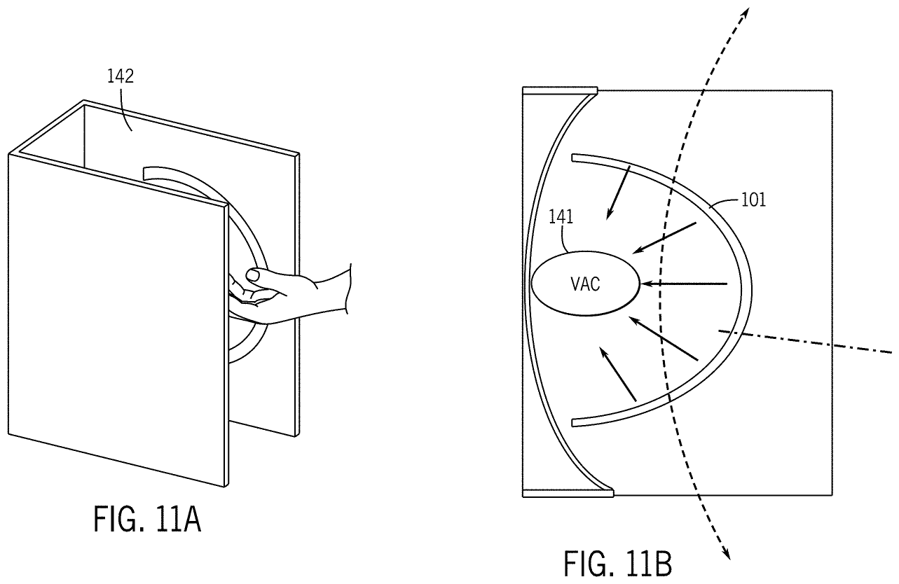

FIGS. 11A and 11B illustrate another arrangement of air knives 101 for a hand dryer. A curved air knife 101 may be angled so that all parts of the air knife 101 are pointed toward the vacuum 141. Air blades may have a compound angle in the arc (approximately 30 degrees from the horizontal) with an air knife velocity of 100-200 m/s. The vacuum 141 may include a flow rate of about 50 cubic feet per minute (CFM) and may match or exceed the flow rate of the air knifes in aggregate.

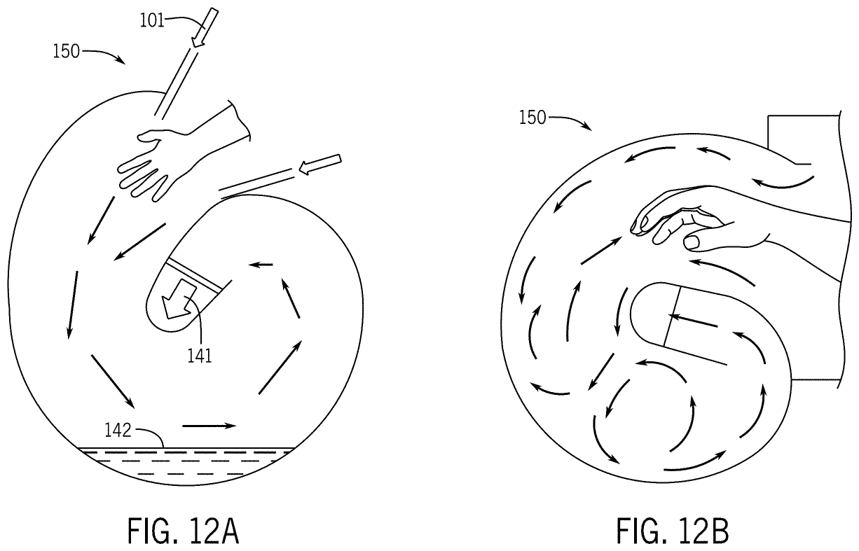

FIGS. 12A and 12B illustrate a circular air duct 150 with a vacuum source 141 for a hand dryer. One or more air knives 101 are mounted at the inlet of the circular air duct 150. The user's hand or hands are placed at the inlet of the circular air duct 150. The circular air duct 150 provides a circular path for the air, water, and suspended particles. The water may be collected at the collection device 142 at the bottom of the circular air duct 150. A drain may be provided to empty the collection device 142.

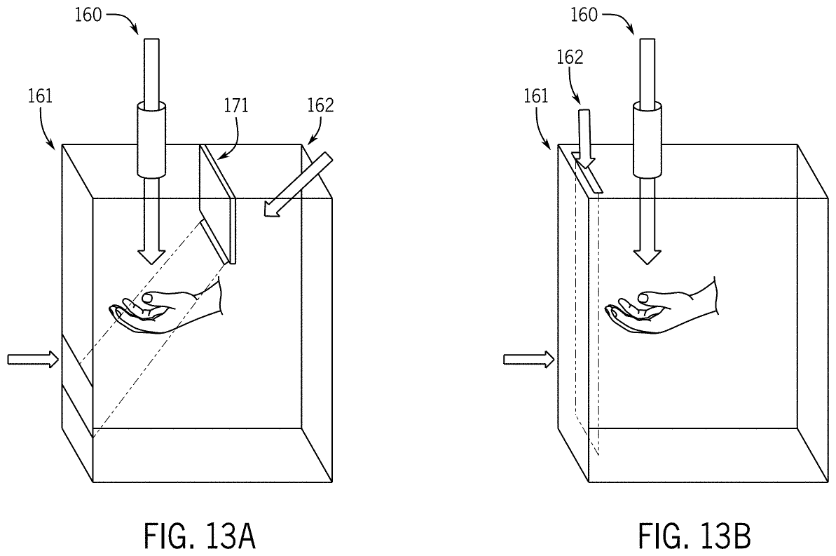

FIGS. 13A and 13B illustrate another embodiment of a hand dryer with a drying air source 160, air curtains 162 and walls 161. The air curtains 162 are thin jets of air (e.g., as provided by air knives) that provide air walls within the hand dryer. FIG. 13A illustrates that the air curtain 162 is positioned behind the user's hand(s) and configured to wash bacteria, water, and other particles away from the user and toward the wall 161. An interior wall 171 may direct the air curtain 162.

FIG. 13B illustrates that the air curtain 162 provides a vertical barrier between the user's hand(s) and the wall 161. In this way, any water, bacteria, or other particles being blows toward the wall 161 will not be reflected off of the wall. The air curtain 162 prevents the bacteria from getting blown off the wall from the hand dryer.

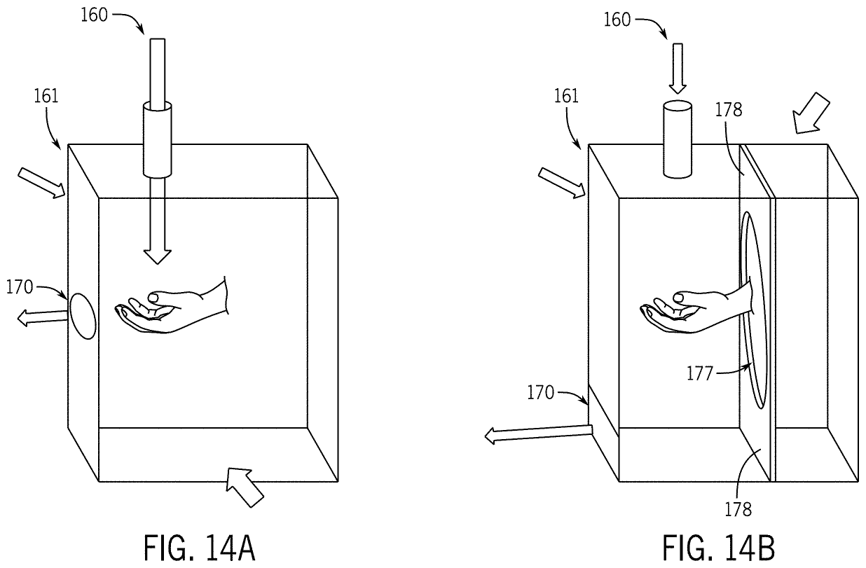

FIGS. 14A and 14B illustrate another embodiment of a hand dryer coupled with a vacuum 170. The vacuum 170 may be positioned behind the hand (e.g., upstream of opening 177), at a level substantially similar to the hand (e.g., shown in FIG. 14A), and/or at any lower position in the hand drying chamber (e.g., shown in FIG. 14B). In FIG. 14A a vacuum 170 is placed in the wall 161 to pull the air, water, bacteria, or other particles out of the hand dryer and into the wall 161. The exhaust may be vented outside of the building, to another room, into a passage in the wall, a ventilation system or a specified chamber. In FIG. 14B the vacuum 170 is downstream to pull air, water, bacteria, or other particles down and away from the user's hand(s) and into the wall 161. The vacuums 170 are configured to remove the aerosols generated by hand drying. In addition or in the alternative, a blower or fan may be upstream of the chamber. Additional, different, or fewer components may be included.

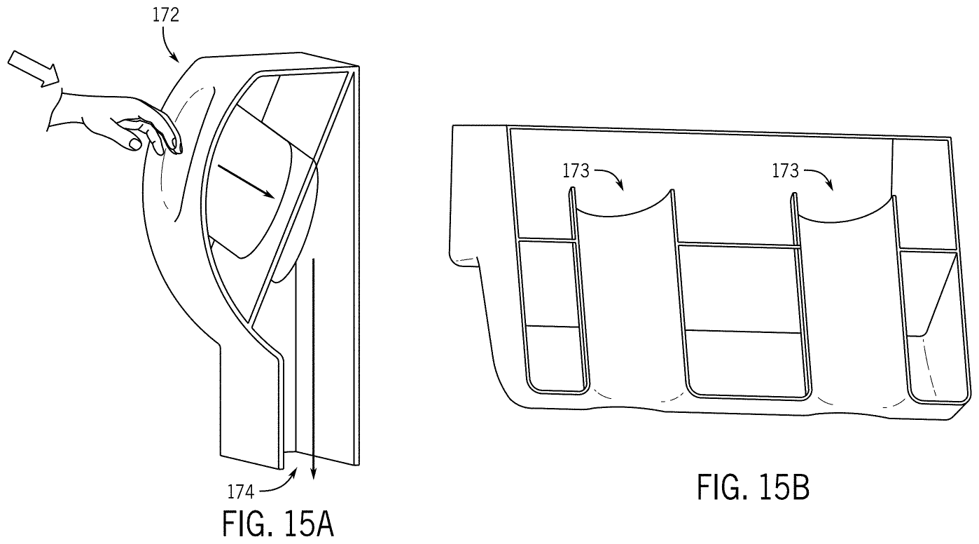

FIGS. 15A and 15B illustrate another embodiment of a hand dryer 172 having various vacuum positions. The embodiment of FIGS. 15A-B includes dual ducts 173. Each duct 173 corresponds to a different hand. The user's hands are placed into the ducts past the air knives (i.e., deeper into the ducts 173 than the air knives). The exhaust 174 provides a path for the air, water, and aerosols that are blown from the user hand(s) to escape through the bottom of the hand dryer 172. The exhaust 174 may empty into a space below the floor. The exhaust 174 may connect to a ventilation system. In some examples, each of the ducts 173 is connected to a separate exhaust path. In some examples, the ducts 173 are combined (connected) into a single exhaust path.

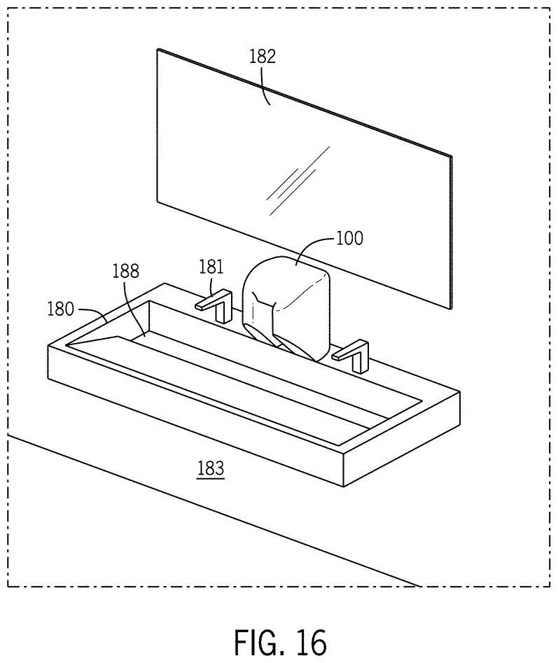

FIG. 16 illustrates an example hand dryer 100 according to any of the embodiments herein mounted in association with a sink 180 including one or more faucets 181 and a drain 188 and/or in association with mirror 182. The hand dryer 100 may be mounted above the sink 180 but still providing an open space 183 (e.g., for mopping, user's feet, a wheelchair, etc.). Additional, different, or fewer components may be included.

In one example, the operation of the hand dryer 100 is tied to the faucets 181. For example, the faucets 181 may be actuated (e.g., turned on) through a proximity or motion sensor. The hand dryer 100 may be turned on after a predetermined time (e.g., 10 seconds, 20 seconds, 30 seconds). The predetermined time may be selected to encourage handwashing for that amount of time.

The faucets 181 dispense water that empties through drain 188. The drain of the hand dryer (e.g., drain 115) may be fluidly coupled to the drain 188. Thus, the water that drains from the sink 180 and the hand dryer 100 may be connected with a T or other coupling device behind or underneath the sink 180.

In addition, the hand dryer 100 may be connected to an exhaust for the air through the wall behind the sink 180. Thus, the hand dryer 100 includes a water drain and an air exhaust that may be located at least partially in the sink 180 and/or the supporting wall.

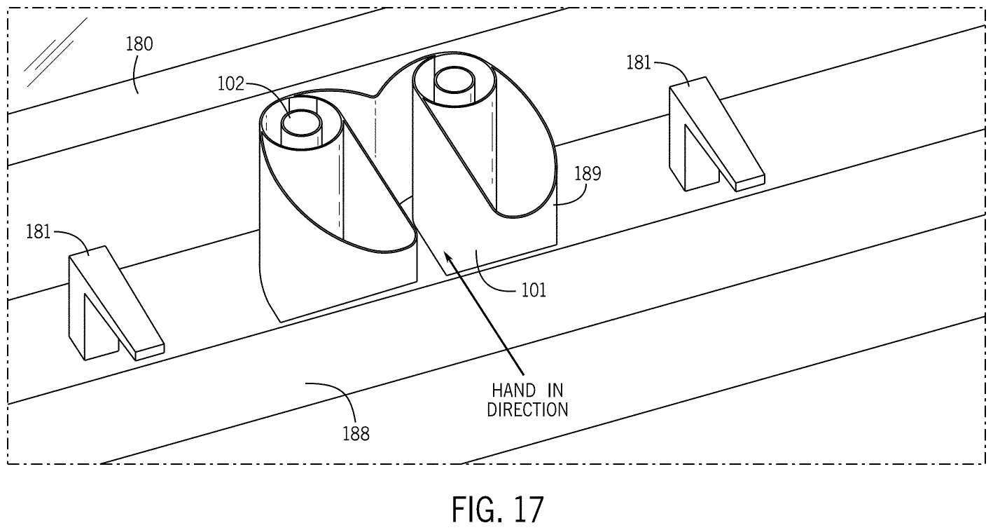

FIG. 17 illustrates a cutaway view of the example hand dryer 100 of FIG. 16. FIG. 17 illustrates that the cyclones 102 and/or housing 189 are mounted between faucets 181 on the sink 180. The air knives 101 may be mounted with the housing 189 so that only an opening is visible. The opening may be slanted down and inward into the hand drying duct of the housing 189.

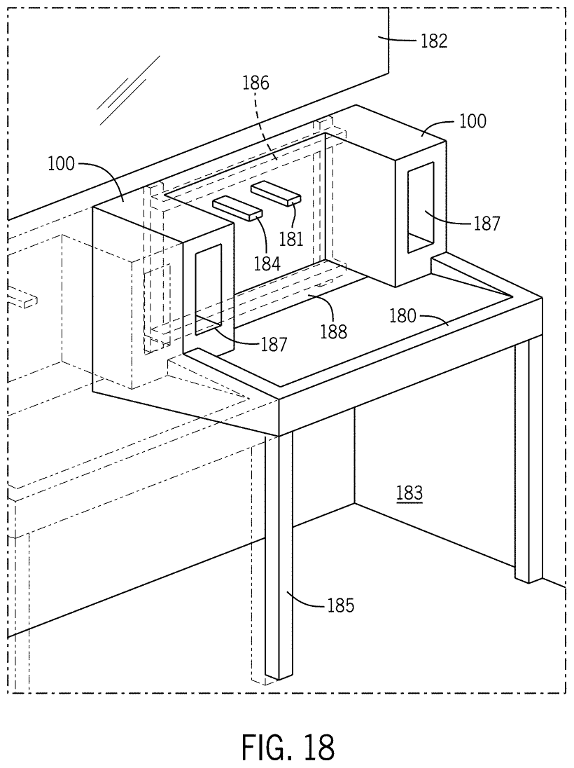

FIG. 18 illustrates multiple hand dryers 100 according to any of the embodiments herein integrated with a sink 180. In this example a single housing may support or be coupled to both the sink 180 and the hand dryer 100. The housing may also support the faucet 181 and a soap dispenser 184. A leg or support 185 may provide support from the floor for the hand dryers 100 and the sink 180. A mount 186 may support the housing including the sink 180 and the hand dryers through a connected with the wall. The mount 186 may be located between the wall and the housing supporting the sink 180. Multiple sinks 180 may be mounted adjacent to one another. In some examples, each sink 180 is associated with a single-hand dryer 100 on each side. In some examples, adjacent sinks 180 share the single-hand dryer 100 between them. Additional, different, or fewer components may be included.

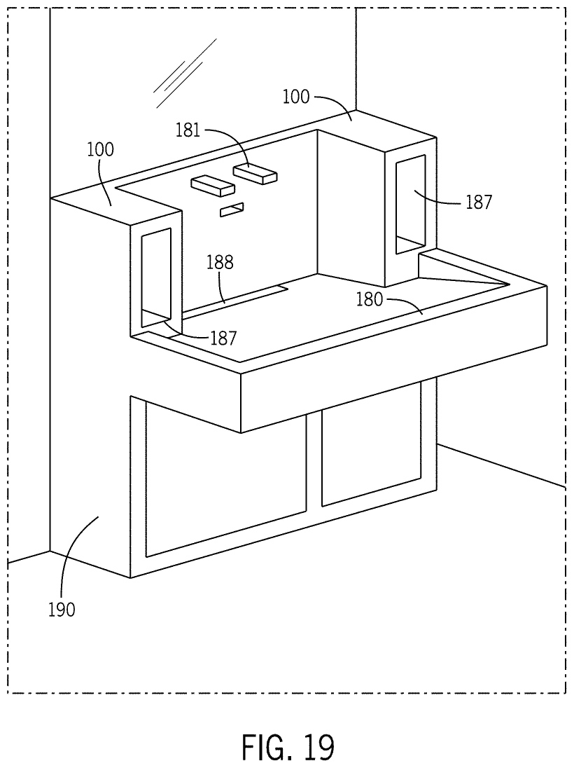

FIG. 19 illustrates multiple hand dryers 100 according to any of the embodiments herein integrated with a sink 180 and cabinet 190. The air exhaust from the hand dryers 100 may pass through the cabinet 190 to give access for maintenance. Similarly, the water drain for the sink 180 and the hand dryer 100 may pass through the cabinet 190 to give access for maintenance. An electrical connection may be provided within the cabinet 190. The electrical connection may provide power to an automatic valve of the faucet 181 and/or the fan of the hand dryers 100.

FIG. 20 illustrates an example control system 301 for any of the dryer systems of FIGS. 1-19. The control system 400 may implement the controller 10 in other examples. The control system 400 may include a processor 300, a memory 352, and a communication interface 353 for interfacing with devices or to the internet and/or other networks 346. In addition to the communication interface 353, a sensor interface may be configured to receive data from the sensors described herein or data from any source described herein. The components of the control system 400 may communicate using bus 348. The control system 400 may be connected to a workstation or another external device (e.g., control panel) and/or a database for receiving user inputs, system characteristics, and any of the values described herein.

The control system 400 may include a sensor 12 configured to generate sensor data for an object in vicinity to the sensor, and/or timer 11 configured to measure an elapsed time period. The processor 300 is configured to generate instructions to operate the hand dryer (e.g., turn on a fan) to move air through the at least one cyclone in response to the sensor data or the elapsed time period.

Optionally, the control system 400 may include an input device 355 and/or a sensing circuit in communication with any of the sensors. The sensing circuit receives sensor measurements from sensor 12 as described above. The input device 355 may include a switch (e.g., actuator), a touchscreen coupled to or integrated with, a keyboard, a remote, a microphone for voice inputs, a camera for gesture inputs, and/or another mechanism.

Optionally, the control system 400 may include a drive unit 340 for receiving and reading non-transitory computer media 341 having instructions 342. Additional, different, or fewer components may be included. The processor 300 is configured to perform instructions 342 stored in memory 352 for executing the algorithms described herein. A display 350 may be supported by any of the components described herein. The display 350 may be combined with the user input device 355.

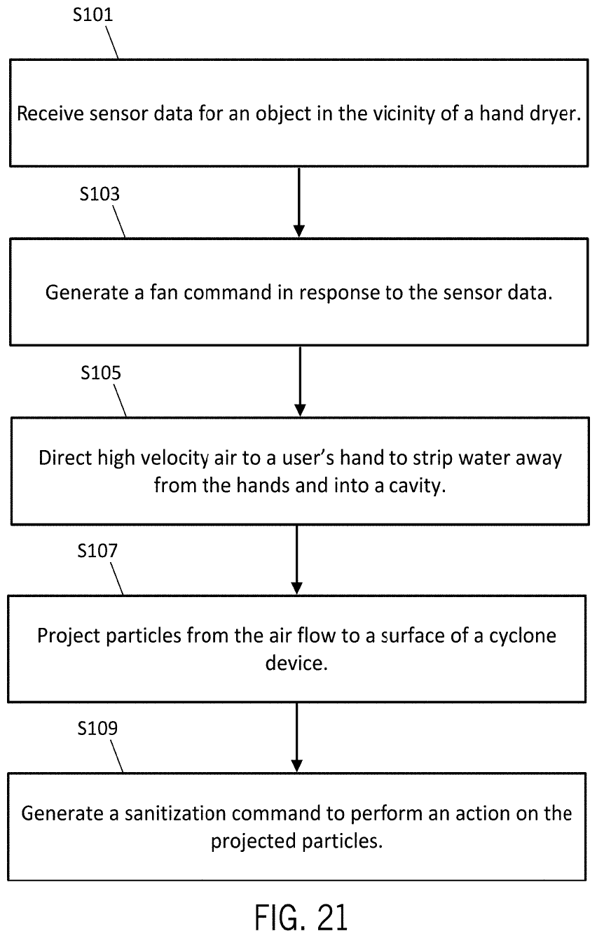

FIG. 21 illustrates a flow chart for the control system 400 of FIG. 19. The acts of the flow chart may be performed by any combination of the control system 400, the network device, or the server. Additional, different or fewer acts may be included.

At act S101, the processor 300 may receive sensor data from the sensor 12. The sensor data is indicative of presence of an object in vicinity of the hand dryer 100. The sensor 12 may detect the presence of one or more hands within a drying duct of the hand dryer 100. The sensor data may indicate that a faucet has been used. The sensor data may indicate the presence of a user near the hand dryer 100.

At act S103, the processor 300 generates a fan command in response to the sensor data. The fan command instructs a fan to operate or turn on (i.e., propel air) to an air knife. The air knife may provide a narrow path for the air that increases the velocity of the air and expels the air through at least one opening. At act S105, the flow of the air knife has an increased velocity and is directed to a user's hand where water (e.g., including particles or aerosols) is mechanically stripped from the hands into a ducted cavity towards at least one cyclone chamber.

At act S107, the air flow is subsequently directed to at least one cyclone chamber where it follows at least a partially circular path or circuitous path to project particles to a surface of the cyclone chamber. Some of the air exits the cyclone chamber through an exhaust path. At least some of the water exits the cyclone chamber through a drain. In some examples, a portion of the water may exit with the air through the exhaust path.

At act S109, the processor 300 generates a sanitization command in response to the sensor data to perform a sanitization, disinfection, or other cleaning action on the projected particles. The sanitation command may cause an ultraviolet line to irradiate the at least one cyclone chamber (e.g., including particles or aerosols adhered to the inner surface of the at least one cyclone chamber). The sanitation command may cause a misting generator to generate a mist including a chemical (e.g., hydrogen peroxide) in the at least one cyclone chamber. The sanitation command may cause an ozone generator to release ozone in the at least one cyclone chamber. Any combination of these disinfection techniques may be used.

Processor 300 may be a general purpose or specific purpose processor, an application specific integrated circuit (ASIC), one or more programmable logic controllers (PLCs), one or more field programmable gate arrays (FPGAs), a group of processing components, or other suitable processing components. Processor 300 is configured to execute computer code or instructions stored in memory 352 or received from other computer readable media (e.g., embedded flash memory, local hard disk storage, local ROM, network storage, a remote server, etc.). The processor 300 may be a single device or combinations of devices, such as associated with a network, distributed processing, or cloud computing.

Memory 352 may include one or more devices (e.g., memory units, memory devices, storage devices, etc.) for storing data and/or computer code for completing and/or facilitating the various processes described in the present disclosure. Memory 352 may include random access memory (RAM), read-only memory (ROM), hard drive storage, temporary storage, non-volatile memory, flash memory, optical memory, or any other suitable memory for storing software objects and/or computer instructions. Memory 352 may include database components, object code components, script components, or any other type of information structure for supporting the various activities and information structures described in the present disclosure. Memory 352 may be communicably connected to processor 300 via a processing circuit and may include computer code for executing (e.g., by processor 300) one or more processes described herein. For example, memory 298 may include graphics, web pages, HTML files, XML files, script code, shower configuration files, or other resources for use in generating graphical user interfaces for display and/or for use in interpreting user interface inputs to make command, control, or communication decisions.

In addition to ingress ports and egress ports, the communication interface 353 may include any operable connection. An operable connection may be one in which signals, physical communications, and/or logical communications may be sent and/or received. An operable connection may include a physical interface, an electrical interface, and/or a data interface. The communication interface 353 may be connected to a network. The network may include wired networks (e.g., Ethernet), wireless networks, or combinations thereof. The wireless network may be a cellular telephone network, an 802.11, 802.16, 802.20, or WiMax network, a Bluetooth pairing of devices, or a Bluetooth mesh network. Further, the network may be a public network, such as the Internet, a private network, such as an intranet, or combinations thereof, and may utilize a variety of networking protocols now available or later developed including, but not limited to TCP/IP based networking protocols.

While the computer-readable medium (e.g., memory 352) is shown to be a single medium, the term “computer-readable medium” includes a single medium or multiple media, such as a centralized or distributed database, and/or associated caches and servers that store one or more sets of instructions. The term “computer-readable medium” shall also include any medium that is capable of storing, encoding, or carrying a set of instructions for execution by a processor or that cause a computer system to perform any one or more of the methods or operations disclosed herein.

In a particular non-limiting, exemplary embodiment, the computer-readable medium can include a solid-state memory such as a memory card or other package that houses one or more non-volatile read-only memories. Further, the computer-readable medium can be a random access memory or other volatile re-writable memory.

Additionally, the computer-readable medium can include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored. The computer-readable medium may be non-transitory, which includes all tangible computer-readable media.

In an alternative embodiment, dedicated hardware implementations, such as application specific integrated circuits, programmable logic arrays and other hardware devices, can be constructed to implement one or more of the methods described herein. Applications that may include the apparatus and systems of various embodiments can broadly include a variety of electronic and computer systems. One or more embodiments described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

The illustrations of the embodiments described herein are intended to provide a general understanding of the structure of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

While this specification contains many specifics, these should not be construed as limitations on the scope of the invention or of what may be claimed, but rather as descriptions of features specific to particular embodiments of the invention. Certain features that are described in this specification in the context of separate embodiments can also be implemented in combination in a single embodiment.

Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

It is intended that the foregoing detailed description be regarded as illustrative rather than limiting and that it is understood that the following claims including all equivalents are intended to define the scope of the invention. The claims should not be read as limited to the described order or elements unless stated to that effect. Therefore, all embodiments that come within the scope and spirit of the following claims and equivalents thereto are claimed as the invention.

What is claimed is:

1. An apparatus comprising:
a fan;
a duct;
at least one air knife configured to direct air from the fan and configured to dry one or more hands and push water from the one or more hands into the duct; and
at least one cyclone device to provide at least a partially circuitous path for the air and the water from the one or more hands.

2. The apparatus of claim 1, wherein particles expelled from the air and water adhere to the at least one cyclone device.

3. The apparatus of claim 1, wherein the at least one cyclone device comprises an inner channel and an outer channel.

4. The apparatus of claim 1, wherein the at least one air knife includes a first air knife and a second air knife each configured to direct air from the fan to dry a single hand and push water from the single hand to the duct.

5. The apparatus of claim 1, further comprising:
a drain path in communication with the at least one cyclone device to drain the water from the at least one cyclone device.

6. The apparatus of claim 1, further comprising:
a light configured disinfect air and water in the at least one cyclone device.

7. The apparatus of claim 1, further comprising:
a filter configured to remove particles from the air.

8. The apparatus of claim 7, wherein the filter is upstream of the air knife or coupled to the duct.

9. The apparatus of claim 7, wherein the filter is a room filter configured to filter air in a vicinity of the apparatus.

10. The apparatus of claim 1, further comprising:
an exhaust path connected to the at least one cyclone device and configured to move the air away from the apparatus.

11. The apparatus of claim 1, further comprising:
a disinfectant dispenser configured to provide a disinfectant to the at least one cyclone device.

12. The apparatus of claim 1, further comprising:
a sensor configured to generate sensor data for an area in vicinity to the sensor;
a timer configured to measure an elapsed time period; and
a controller configured to operate the fan to move air through the at least one cyclone device in response to the sensor data or the elapsed time period.

13. The apparatus of claim 12, wherein a disinfectant is provided to the at least one cyclone device in response to the sensor data or the elapsed time period.

14. A control system for operation of a hand dryer, the control system comprising:
a sensor configured to generate sensor data for an object in vicinity to the hand dryer;
a timer configured to measure an elapsed time period; and
a controller configured to operate a fan to move air through the hand dryer in response to the sensor data or the elapsed time period.

15. The control system of claim 14, wherein the controller operates in a low flow rate mode to clean air in a room including the hand dryer.

16. The control system of claim 14, wherein the controller operates in a sanitation mode to release a sanitizer into the hand dryer.

17. The control system of claim 14, wherein the controller is configured to operate the fan in response to the sensor data and stop the fan in response to the elapsed time period.

18. A hand dryer comprising:
a left ducted cavity;
a right ducted cavity;
at least one first air knife angled toward the left ducted cavity;
at least one second air knife angled toward the right ducted cavity; and
an expansion cavity coupled to the left ducted cavity and the right ducted cavity and configured to slow down a flow of air from the at least one first air knife and the at least one second air knife, wherein the expansion cavity is configured to collect water droplets or particles suspended in the flow of air.

19. The hand dryer of claim 18, further comprising:
a pressure chamber; and
a fan coupled to the pressure chamber, wherein the pressure chamber provides the flow of air from the fan to the at least one first air knife and at least one second air knife.

20. The hand dryer of claim 18, further comprising:
a sanitization device configured to sanitize, disinfect, or clean the water droplets or particles collected from the flow of air.
